(19) United States
(12) Patent Application Publication
(10) Pub. No.: US 2019/0174694 A1
(43) Pub. Date: Jun. 13, 2019

(54) Ornamental Plant Displaying Compacted Plant Growth

(71) Applicant: Dümmen Group B.V., De Lier (NL)

(72) Inventor: Chunting Lang, De Lier (NL)

(21) Appl. No.: 16/309,650

(22) PCT Filed: Jun. 22, 2017
(86) PCT No.: PCT/EP2017/065455

§ 371 Date: Dec. 13, 2018

(30) Foreign Application Priority Data
Jun. 27, 2016 (NL) 2017054

(51) Int. Cl. A01H 5/02; A01H 1/04; A01H 6/32

(52) U.S. Cl. CPC A01H 5/02; A01H 6/324; A01H 1/04

(57) ABSTRACT

Provided herein are ornamental plants displaying compacted growth or having the phenotype compacted plant growth and methods for providing plants displaying compacted plant growth. Specifically, the ornamental plants displaying compacted plant growth without use of plant growth regulators during cultivation are vegetatively propagated from a first ornamental plant displaying compacted plant growth without use of plant growth regulators during cultivation, said first ornamental plant obtained by inbred breeding of at least 2 generations of a second ornamental plant not displaying compacted plant growth without use of plant growth regulators during cultivation and said ornamental plants have a heterozygosity percentage being decreased by at least 20% as compared to the heterozygosity percentage of said second ornamental plant not displaying compacted plant growth without use of plant growth regulators during cultivation.

The present invention relates to ornamental plants displaying compacted growth or, formulated differently, having the phenotype compacted plant growth. The present invention further relates to a method for providing plants displaying compacted plant growth and to the use of inbreeding, such as by selfing, to obtain ornamental plants with compacted plant growth.

The ornamental plant Kalanchoe is a commercially important indoor pot plant. Currently commercial varieties are obtained through crossing of highly heterozygous parents and clonal propagation. These varieties show high vigor and therefore growth regulators need to be applied in order to achieve a more compact plant shape, which is commercially desired. However, growth regulators are increasingly being banned by regulators. For instance, the growth regulator TILT (Syngenta) is banned in the USA, Canada, Germany and Sweden.

The genus of Kalanchoe belongs to the Crassulaceae family. This genus comprises ˜140 different species (B. Descoings et. 2003) and many species of this genus and interspecific hybrids thereof are cultivated for ornamental use. Within these species there is great variation with respect to commercially important characters such as flower shape, flower color, flower size, number of flower petals, leaf morphology and plant architecture.

The vigor of all current commercial varieties of Kalanchoe needs to be controlled through the use of plant growth regulators (PGRs), in order to keep a compact plant shape. The timing of PGR application is very important for the shape of end products but the correct moment of application is influenced by temperature. This means that it is difficult for a grower to apply PGR at the correct moment. Incorrect PGR application regularly leads to economic loss during cultivation.

It is therefore also commercially interesting to breed Kalanchoe plants in which plants can be grown without PGRs. It has been demonstrated that this can be achieved by transforming K. blossfeldiana with A. rhizogenes, resulting in expression of Rol gene (B. Christensen et al. 2008; H. Liitken et al. 2012; EP 2698432 A1; U.S. Pat. No. 9,253,952; US20140053297; US20160032311)). However, the drawback of this method is that the plants may not keep compact growth over many generations. Also, commercialization of bacterially infected plants is undesirable and may be noncompliant with national and regional plant health regulations.

Considering the above, there is a need in the art for generating ornamental plants such as Kalanchoe that can grow in a compact shape without application of growth regulators.

It is an object of the present invention, amongst objects to meet the above need in the art.

According to the present invention, the above object, amongst other objects, is met by plants, methods and uses as outlined in the appended claims.

Specifically, the above object of the present invention, amongst other objects, is met by providing ornamental plants displaying compacted plant growth without use of plant growth regulators during cultivation;
said ornamental plant displaying compacted plant growth without use of plant growth regulators during cultivation is vegetatively propagated, such as clonal propagation, from a first ornamental plant displaying compacted plant growth without use of plant growth regulators during cultivation said first ornamental plant is obtainable by inbred breeding of at least 2 generations of a second ornamental plant not displaying compacted plant growth without use of plant growth regulators during cultivation; and
said ornamental plant is selected from plants belonging to a family selected from the group consisting of Liliaceae, Araceae, Solanaceae, Euphorbiaceae, Geraniaceae, Asteraceae, Orchidaceae, Rosaceae, Caryophyllaceae and Crassulaceae.
said ornamental plant has a heterozygosity percentage being decreased by at least 20% as compared to the heterozygosity percentage of said second ornamental plant not displaying compacted plant growth without use of plant growth regulators during cultivation.

According to the present invention, the decrease in heterozygosity is at least 20% such as at least 25%, 30%, 35%, 40%, 50%, 55%, 60%, 70%, 75%.

The present inventors have surprisingly discovered that by using inbreeding during several generations a stable, i.e. genetically transmittable, compacted growth phenotype can be obtained. Without wishing to be limited to any underlying mechanism, it is believed that an increase in homozygosity in F3 progeny and further, inherently occurring through inbred breeding or selfing, is responsible for the observed phenotype especially considering that the phenotype of compact plant growth is observed in a number of genetic backgrounds.

According to a preferred embodiment, the present displaying compacted plant growth without use of plant growth regulators during cultivation can be stably transmitted to progeny thereof. Formulated differently, progeny of a plant displaying compacted plant growth have the same phenotype with respect to this trait.

According to another preferred embodiment, the present ornamental plant is obtainable by at least 3 generations of inbred breeding (F4), preferably at least 4 (F5), more preferably at least 5 (F6), even more preferably at least 6 (F7) and most preferably at least 7 (F8) such as 8 (F9), 9 (F10), 10 (F11), 11 (F12) or 12 (F13).

According to and especially preferred embodiment, the present ornamental plant is a plant belonging to the family Crassulaceae and more specifically the genus Kalanchoe and is selected from the group consisting of Kalanchoe adelae, Kalanchoe arborescens, Kalanchoe beauverdii, Kalanchoe beharensis, Kalanchoe bentii, Kalanchoe blossfeldiana, Kalanchoe bouvetii, Kalanchoe bracteata, Kalanchoe campanulata, Kalanchoe crenata, Kalanchoe crundallii, Kalanchoe daigremontiana, Kalanchoe delagoensis, Kalanchoe dinklagei, Kalanchoe eriophylla, Kalanchoe farinacea, Kalanchoe fedtschenkoi, Kalanchoe figuereidoi, Kalanchoe flammea, Kalanchoe gastonis, Kalanchoe glaucescens, Kalanchoe gracilipes, Kalanchoe grandidieri, Kalanchoe grandiflora, Kalanchoe hildebrantii, Kalanchoe jongmansii, Kalanchoe kewensis, Kalanchoe laciniata, Kalanchoe laetivirens, Kalanchoe lateritia, Kalanchoe laxiflora, Kalanchoe linearifolia, Kalanchoe longiflora, Kalanchoe luciae, Kalanchoe macrochlamys, Kalanchoe manginii, Kalanchoe marnieriana, Kalanchoe marmorata, Kalanchoe Kalanchoe miniata, Kalanchoe nyikae, Kalanchoe obtusa, Kalanchoe orgyalis, Kalanchoe peltata, Kalanchoe petitiana, Kalanchoe pinnata, Kalanchoe porphyrocalyx, Kalanchoe prolifera, Kalanchoe pubescens, Kalanchoe pumila, Kalanchoe quartiniana, Kalanchoe rhombopilosa, Kalanchoe robusta, Kalanchoe rolandi, Kalanchoe rosei, Kalanchoe rotundifolia, Kalanchoe schizophyila, Kalanchoe serrata, Kalanchoe sexangularis, Kalanchoe streptantha, Kalanchoe suarezensis, Kalanchoe synsepala, Kalanchoe synsepala f. dissecta, Kalanchoe thyrsiflora, Kalanchoe tomentosa, Kalanchoe tubiflora, Kalanchoe uniflora, Kalanchoe velutina and Kalanchoe viguieri; preferably decorative flowering Kalanchoe plants selected from the group consisting of K. blossfeldiana, K. laciniata, K. rotundifolia, K. aromatica, K. pubescens, K. grandiflora, K. citrina, K. ambolensis, K. faustii, K. schumacherii, K. pritwitzii, K. flamniea, K. figueredoi, K. rauhii, K. obtusa, K. pumila, K. marmorata, K. porphyrocalux, K. jongmansii, K. pinnata, K. diagremontiana, K. gracilipes, K. campanulata, K. latisepela, K. coccinea, K. fedtschenkoi, K. tubiflora, K. decumbens, K. manginii, K. orgyalis, K. crenata, K. tomentosa and hybrids thereof.

According to yet another especially preferred embodiment, the present compacted plant growth is compactness expressed as one or more of plant height, inflorescence length and plant width, preferably wherein said plant height, inflorescence length and plant width is reduced in each generation (Fₓ) as compared to the previous generation (Fₓ₋₁) and most preferably wherein the ratio between Fₓ and Fₓ₋₁(Fₓ/Fₓ₋₁) of plant height, inflorescence length and/or plant width is <1.

The present invention relates to methods for providing the present ornamental plants displaying compacted plant growth without use of plant growth regulators during cultivation, the methods comprise the step of:
a) vegetatively propagating a first ornamental plant displaying compacted plant growth without use of plant growth regulators during cultivation said first ornamental plant is obtainable by inbred breeding of at least 2 generations of a second ornamental plant not displaying compacted plant growth without use of plant growth regulators during cultivation.

Preferably, the present methods use in selfing or self-pollination to provide inbreeding.

The present methods, according to another preferred embodiment, comprise least 3 (F4) generations of inbred breeding, preferably at least 4 (F5), more preferably at least 5 (F6), even more preferably at least 6 (F7) and most preferably at least 7 (F8).

According to and especially preferred embodiment, the ornamental plants of the present methods are plants belonging to a family selected from the group consisting of Liliaceae, Araceae, Solanaceae, Euphorbiaceae, Geraniaceae, Asteraceae, Orchidaceae, Rosaceae, Caiyophyllaceae and Crassulaceae, more preferably Kalanchoe such as a Kalanchoe ornamental plant is selected from the group consisting of Kalanchoe adelae, Kalanchoe arborescens, Kalanchoe beauverdii, Kalanchoe beharensis, Kalanchoe bentii, Kalanchoe blossfeldiana, Kalanchoe bouvetii, Kalanchoe bracteata, Kalanchoe campanulata, Kalanchoe crenata, Kalanchoe crundallii, Kalanchoe daigremontiana, Kalanchoe delagoensis, Kalanchoe dinklagei, Kalanchoe eriophylla, Kalanchoe farinacea, Kalanchoe fedtschenkoi, Kalanchoe figuereidoi, Kalanchoe flammea, Kalanchoe gastonis, Kalanchoe glaucescens, Kalanchoe gracilipes, Kalanchoe grandidieri, Kalanchoe grandiflora, Kalanchoe hildebrantii, Kalanchoe jongmansii, Kalanchoe kewensis, Kalanchoe laciniata, Kalanchoe laetivirens, Kalanchoe lateritia, Kalanchoe laxiflora, Kalanchoe linearifolia, Kalanchoe longiflora, Kalanchoe luciae, Kalanchoe macrochlamys, Kalanchoe manginii, Kalanchoe marnieriana, Kalanchoe marmorata, Kalanchoe millottii, Kalanchoe miniata, Kalanchoe nyikae, Kalanchoe obtusa, Kalanchoe orgyalis, Kalanchoe peltata, Kalanchoe petiliana, Kalanchoe pinnata, Kalanchoe porphyrocalyx, Kalanchoe prolifera, Kalanchoe pubescens, Kalanchoe pumila, Kalanchoe quartiniana, Kalanchoe rhombopilosa, Kalanchoe robusta, Kalanchoe rolandi, Kalanchoe rosei, Kalanchoe rotundifolia, Kalanchoe schizophylla, Kalanchoe serrata, Kalanchoe sexangularis, Kalanchoe streptantha, Kalanchoe suarezensis, Kalanchoe synsepala, Kalanchoe synsepala f dissecta, Kalanchoe thyrsiflora, Kalanchoe tomentosa, Kalanchoe tubiflora, Kalanchoe uniflora, Kalanchoe velutina and Kalanchoe viguieri; preferably decorative flowering Kalanchoe plants selected from the group consisting of K. blossfeldiana, K. laciniata, K. rotundifolia, K. aromatica, K. pubescens, K. grandiflora, K. citrina, K. ambolensis, K. faustii, K. schumacherii, K. pritwitzii, K. flammea, K. figueredoi, K. rauhii, K. obtusa, K. pumila, K. marmorata, K. porphyrocalux, K. jongmansii, K. pinnata, K. diagremontiana, K. gracilipes, K. campanulata, K. latisepela, K. coccinea, K. fedtschenkoi, K. tubiflora, K. decumbens, K. manginii, K. orgyalis, K. crenata, K. tomentosa and hybrids thereof.

The present invention also relates to the use of inbreeding for at least 2 generations for providing the above ornamental plants displaying compacted plant growth without use of plant growth regulators during cultivation.

The present invention further relates to Kalanchoe plants displaying compacted plant growth without use of plant growth regulators during cultivation obtainable by inbred breeding of at least 2 generations of a Kalanchoe plant not displaying compacted plant growth without use of plant growth regulators during cultivation; said Kalanchoe plants have a heterozygosity percentage being decreased by at least 20%, such as at least 25%, 30%, 35%, 40%, 50%, 55%, 60%, 70%, 75%, as compared to the heterozygosity percentage of said second Kalanchoe plant not displaying compacted plant growth without use of plant growth regulators during cultivation.

The above Kalanchoe plants are preferably a Kalanchoe plant selected from the group consisting of Kalanchoe adelae, Kalanchoe arborescens, Kalanchoe beauverdii, Kalanchoe beharensis, Kalanchoe bentii, Kalanchoe blossfeldiana, Kalanchoe bouvetii, Kalanchoe bracteata, Kalanchoe campanulata, Kalanchoe crenata, Kalanchoe crundallii, Kalanchoe daigremontiana, Kalanchoe delagoensis, Kalanchoe dinklagei, Kalanchoe eriophylla, Kalanchoe farinacea, Kalanchoe fedtschenkoi, Kalanchoe figuereidoi, Kalanchoe flammea, Kalanchoe gastonis, Kalanchoe glaucescens, Kalanchoe gracilipes, Kalanchoe grandidieri, Kalanchoe grandiflora, Kalanchoe hildebrantii, Kalanchoe jongmansii, Kalanchoe kewensis, Kalanchoe laciniata, Kalanchoe laetivirens, Kalanchoe lateritia, Kalanchoe laxflora, Kalanchoe linearifolia, Kalanchoe longiflora, Kalanchoe luciae, Kalanchoe macrochlamys, Kalanchoe manginii, Kalanchoe marnieriana, Kalanchoe marmorata, Kalanchoe millottii, Kalanchoe miniata, Kalanchoe nyikae, Kalanchoe obtusa, Kalanchoe orgyalis, Kalanchoe peltata, Kalanchoe petitiana, Kalanchoe pinnata, Kalanchoe porphyrocalyx, Kalanchoe prolifera, Kalanchoe pubescens, Kalanchoe pumila, Kalanchoe quartiniana, Kalanchoe rhombopilosa, Kalanchoe robusta, Kalanchoe rolandi, Kalanchoe rosei, Kalanchoe rotundifolia, Kalanchoe schizophylla, Kalanchoe serrata, Kalanchoe sexangularis, Kalanchoe streptantha, Kalanchoe suarezensis, Kalanchoe synsepala, Kalanchoe synsepala f. dissecta, Kalanchoe thyrsiflora, Kalanchoe tomentosa, Kalanchoe tubiflora, Kalanchoe uniflora, Kalanchoe velutina and Kalanchoe viguieri; preferably decorative flowering Kalanchoe plants selected from the group consisting of K. blossfeldiana, K. laciniata, K. rotundifolia, K. aromatica, K. pubescens, K. grandiflora, K. citrina, K. ambolensis, K. faustii, K. schumacherii, K. pritwitzii, K. flammea, K. figueredoi, K. rauhii, K. obtusa, K. pumila, K. marmorata, K. porphyrocalux, K. jongmansii, K. pinnata, K. diagremontiana, K. gracilipes, K. campanulata, K. latisepela, K. coccinea, K. fedtschenkoi, K. tubiflora, K. decumbens, K. manginii, K. orgyalis, K. crenata, K. tomentosa and hybrids thereof.

According to an especially preferred embodiment, the above Kalanchoe plants are obtainable by at least 3 generations of inbred breeding, preferably at least 4, more preferably at least 5, even more preferably at least 6 and most preferably at least 7.

The present invention also relates to the use of the present Kalanchoe plants for providing a Kalanchoe plant, preferably displaying compacted plant growth without use of plant growth regulators during cultivation. The present use can comprises vegetative or generative propagation.

The present invention will be further detailed in the examples below. In the example reference is made to Figures wherein:

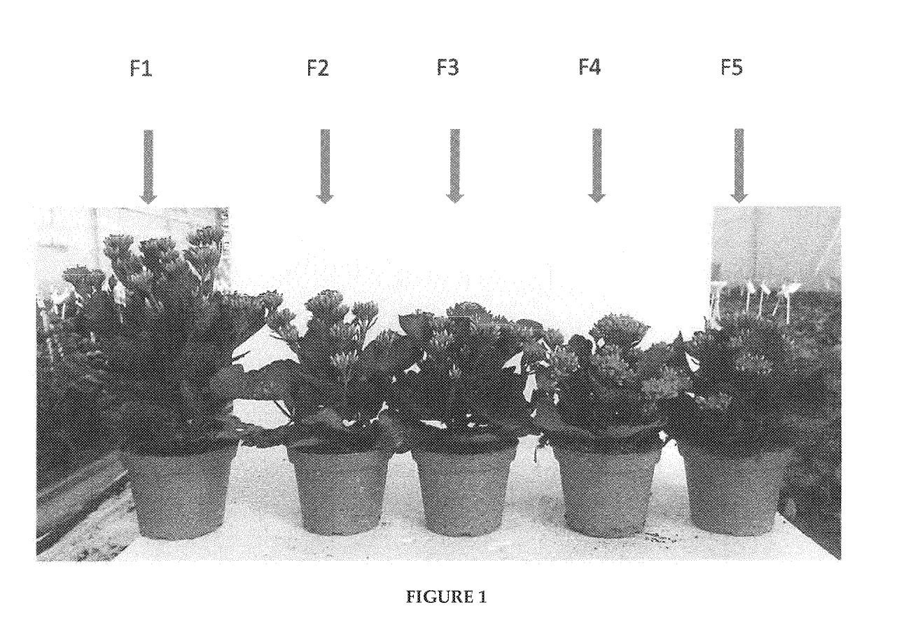

FIG. 1: shows inbred series of the variety “Paso” showing the effect of inbreeding on compacted plant growth;

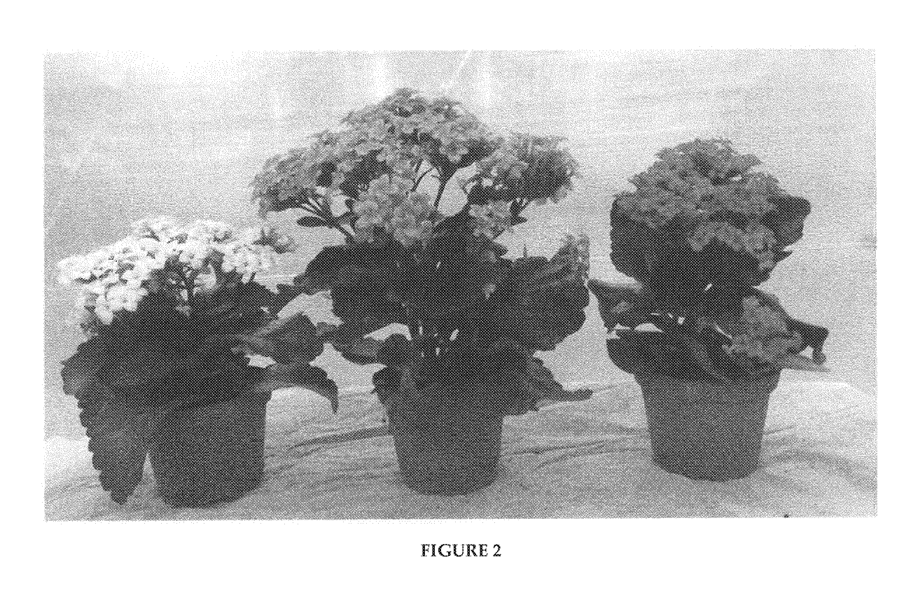

FIG. 2: shows that crossing of two genetically different compact inbreeding lines results in a plant without compacted plant growth;

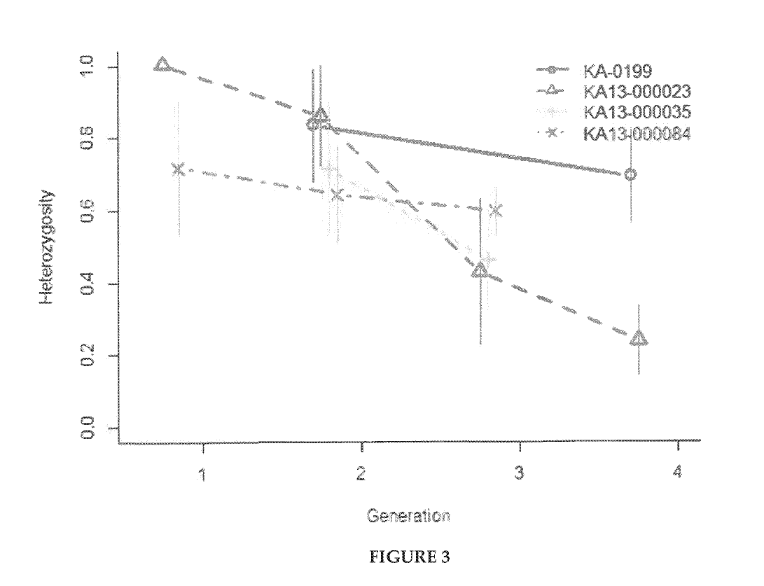

FIG. 3: Lineage-specific trends of heterozygosity as a function of generations of inbreeding (bars denote standard error).

### EXAMPLE 1

Kalanchoe Plants Displaying Compacted Plant Growth

### Introduction

Kalanchoe is a commercially important indoor pot plant. Currently commercial varieties are obtained through crossing of highly heterozygous parents and clonal propagation. These varieties show high vigor and therefore growth regulators need to be applied in order to achieve a more compact plant shape, which is commercially desired. However, growth regulators are increasingly being banned by regulators. For instance, the growth regulator TILT (Syngenta) is banned in the USA, Canada, Germany and Sweden. Here a novel method is described for obtaining Kalanchoe varieties that grow into a compact shape without application of growth regulator by performing inbreeding for at least 2 generations.

### Material and Methods

Varieties of K. blossfeldiana and offspring of interspecific crosses within the Kalanchoe genus were self-pollinated and the resulting seeds were obtained. After sowing, seedlings composing the F2 generation were grown towards maturity (first flowers open) and selected for agronomic traits of importance and for flower color and morphology. Flowers of selected plants were again self-pollinated and the seeds were sown. These seedlings composed the F3 generation and were grown and selected in the same manner as the F2 generation. The process is repeated until a minimum of the F3 generation. The inbreeding process is deemed complete when it results in at least one single seedling that is compact.

Compactness was determined as follows:
plant height, measured from soil surface up to the highest point of a mature plant,
plant width, measured as the largest distance between the tips of leaves on opposing sides of the plants, and
inflorescence length, measured from the highest rosette leaf stem and the highest point of a mature plant

### Results

Inbred series composed of the F1 (original hybrid), F2, F3, F4 and following generations were created by repeated selfing and selection from the commercial varieties ‘Paso’ and ‘Swan’. The selected seedlings from every inbreeding generation were also propagated vegetatively. Cuttings of identical size were made from all generations at the same moment, rooted and grown under identical conditions (rooted for 3 weeks at 14 hrs. day length, grown for 9 weeks at 10 hrs day length in a greenhouse in 10 cm pots). Overall, we were surprised to see selections that displayed a decrease at comparable rates of plant height and inflorescence length. The ratio between both parameters remained quite constant in successive generations, like plant width. The photo shown in FIG. 1 was made 12 weeks after cuttings were rooted and shows the effect of successive generations of inbreeding. From the F3 generation onwards, plants are sufficiently compact to be grown without the need for application of growth regulator (Table 1). Cuttings from the F5 originating from Paso were crossed with a similarly compact F5 inbreeding line from the variety “Swan” (FIG. 2). The resulting F1 hybrid restores original vigor. This result proves that the compactness of both F5 lines is not the result of recurrent selection for compact plants, but is instead caused by inbreeding depression (which is negated by the F5×F5 cross to result in a vigorous hybrid).

TABLE 1
Plant height, inflorescence length and plant
width data from inbreds of ‘Paso’ and ‘Swan’
(in cm ± standard deviation)

| Generation | Plant height | Inflorescence length | Plant width |
|---|---|---|---|
| Paso (F1) | 27.5 ± 0.0 | 20.2 ± 0.0 | 21.0 ± 0.0 |
| F2 | 20.0 ± 0.0 | 14.0 ± 0.0 | 18.0 ± 0.0 |
| F3 | 18.0 ± 4.2 | 12.5 ± 2.1 | 18.0 ± 9.9 |
| F4 | 17.0 ± 0.0 | 13.0 ± 0.0 | 15.8 ± 3.6 |
| F5 | 14.5 ± 0.6 | 11.6 ± 0.3 | 18.3 ± 4.8 |
| Swan (F1) | 21.5 ± 0.0 | 16.0 ± 0.0 | 21.0 ± 0.0 |
| F2 | 16.0 ± 0.0 | 11.0 ± 0.0 | 18.0 ± 0.0 |
| F3 | 15.7 ± 0.8 | 11.2 ± 0.9 | 18.0 ± 1.1 |
| F4 | 15.4 ± 1.4 | 10.6 ± 0.7 | 15.8 ± 1.3 |
| F5 | 13.0 ± 1.1 | 8.8 ± 0.7 | 18.3 ± 2.0 |

### Discussion

In ornamental plants this is a highly novel approach because inbreeding often results in severely stunted plants that are of no commercial value due to inbreeding depression. By balancing inbreeding with selection we avoid the effects of inbreeding on fitness while exploiting the effect on plant height in order to generate compact plants with commercial value.

### EXAMPLE 2

Homozygosity of Kalanchoe Inbred Lines Across Generations of Inbreeding

### Introduction

Inbreeding is a process which occurs under natural conditions in nature and under artificial conditions during commercial breeding. One of the known effects of inbreeding is the decrease of genetic diversity and thus increases of the level of homozygosity.

Whereas an increase of homozygosity under inbreeding is generally perceived as a negative effect of inbreeding, we have to our surprise observed positive effects of inbreeding in the breeding of Kalanchoe varieties. We have observed that repeated inbreeding in this succulent crop results in the desirable trait of compact growth. To observe whether this repeated inbreeding is also correlated with the level of heterozygosity, we performed a molecular genetic screen to assess the level of heterozygosity along the process of inbreeding.

### Material & Methods

22 Kalanchoe plants resulting from between 1 and 4 consecutive generations of inbreeding were selected for measurements of the level of heterozygosity. These plants originated from four independent lineages.

Samples were taken from all plants, DNA was extracted and the DNA was submitted to an external service provider for genotyping single nucleotide polymorphisms (SNP) using KASP assays.

SNPs were selected based on high PIC values (Polymorphism Information Content; maximizes the chance of informative markers in Dümmen Orange Kalanchoe germplasm). 7 of these SNPs, which are located on unique contigs and which are polymorphic were used in the analysis. For the 7 SNP results, we analyzed whether heterozygosity changed significantly as a result of repeated inbreeding events using generalized linear mixed model (GLMM) to accommodate the non-independency of within-lineage and within-plant effects.

### Results

When looking at the average level of heterozygosity across generations, we observed a decrease in heterozygosity between 1 and 4 generations of selfing (FIG. 3, Table 2). In general, heterozygosity decreases in all lineages, with the decrease particularly noticeable in lineage KA13-000023. We have used a generalized linear mixed model (GLMM) with a binomial error structure to model the individual heterozygosity (0 for the homozygous state and 1 for the heterozygous state) for all the SNPs simultaneously as a function of generation. Lineage and individual ID were fitted as random effects to account for the non-independency of within-lineage and within-individual observations, as well as accounting for the unequal distribution of lineages over the range of generations.

Heterozygosity decreased significantly (parameter estimate on the log it scale: −0.71, P=0.0009). This translates to an average decline of heterozygosity of 67% at a 95 confidence interval ranging from 56-76% decline, which is somewhat higher than the theoretical prediction of 50% (as heterozygosity declines following 1/2N and N being 1 in the case of self-pollination). Lineage KA13-000023 showed a noticeably sharp decrease in heterozygosity (GLMM using individual plant ID a random effect with a binomial error structure: parameter estimate: −1.52, P=0.009).

The SNP-specific parameter estimates from a GLMM analyzing the within-lineage changes in heterozygosity for each SNP separately, using a binomial error structure where lineage was fitted as a random effect are shown in Table 2. While no significant effect was detected when analyzing the data for each SNP individually, likely as a result of low statistical power, for 7 out of 7 SNPs the estimates supported the expected result of inbreeding: a decrease in heterozygosity in time (Table 2).

TABLE 2
The parameter estimates from a mixed model analyzing the change in SNP-
specific heterozygosity between 1 and 4 generations of inbreeding.

| SNP name | Parameter estimate ^{a} | Standard error | Z value | P |
|---|---|---|---|---|
| kal_t350680 | −4.14599881 | NA | NA | NA |
| kal_t356650 | −0.628621196 | 0.597390439 | −1.052278637 | 0.29267173 |
| kal_t383779 | −1.532854434 | 1.110802376 | −1.379952425 | 0.167601293 |
| kal_t392158 | −15.75071927 | 7.306911286 | −2.155591967 | 0.03111554 |
| kal_t392809 | −4.310998904 | 3.103502631 | −1.389075318 | 0.164809848 |
| kal_t394972 | −0.784840187 | 0.751668129 | −1.044131255 | 0.296424667 |
| kal_t395817 | −2.208268658 | 1.501049725 | −1.47114957 | 0.141250672 |

### CONCLUSIONS

Between 1 and 4 generations of inbreeding, a robust analysis using a mixed model statistical approach revealed that across 4 independent lineages heterozygosity decreased significantly, that the estimated between-generation decrease is more substantial than the theoretical expectation and that the decrease in heterozygosity was dramatically strong in lineage KA13-000023. The overall decline in heterozygosity pattern is not driven by strong SNP-specific patterns, as all 7 SNPs showed the expected pattern of decreasing heterozygosity as a result of repeated episodes of inbreeding.

### REFERENCES

Brian Christensen, Sridevy Sriskandarajah, Margrethe Serek, Renate Müller (2008) Transformation of Kalanchoe blossfeldiana with rol-genes is useful in molecular breeding towards compact growth. Plant Cell Reports, Volume 27, pp 1485-1495.
Henrik Lütken, Sabá Victoria Wallström, Erik Bjorn Jensen, Brian Christensen, Renate Müller (2012) Inheritance of rol-genes from Agrobacterium rhizogenes through two generations in Kalanchoë. Euphytica, Volume 188, pp 397-407.
EP 2698432 A1: Agrobacterium rhizogenes transformation and expression of rol genes in Kalanchoë
U.S. Pat. No. 9,253,952 B2: Agrobacterium rhizogenes transformation and expression of rol genes in Kalanchoë
US 20140053297 A1: Agrobacterium rhizogenes transformation and expression of rol genes in kalanchoë
US 20160032311 A1: Agrobacterium rhizogenes transformation and expression of rol genes in kalanchoë

1. An ornamental plant displaying compacted plant growth without use of plant growth regulators during cultivation;
said ornamental plant displaying compacted plant growth without use of plant growth regulators during cultivation being vegetatively propagated, or derived, from a first ornamental plant displaying compacted plant growth without use of plant growth regulators during cultivation, said first ornamental plant obtained by inbred breeding of at least 2 generations of a second ornamental plant not displaying compacted plant growth without use of plant growth regulators during cultivation;
said ornamental plant is selected from plants belonging to a family selected from the group consisting of Liliaceae, Araceae, Solanaceae, Euphorbiaceae, Geraniaceae, Asteraceae, Orchidaceae, Rosaceae, Caryophyllaceae and Crassulaceae; and
said ornamental plant has a heterozygosity percentage being decreased by at least 20% as compared to the heterozygosity percentage of said second ornamental plant not displaying compacted plant growth without use of plant growth regulators during cultivation.

2. The ornamental plant according to claim 1, wherein said plant is a Kalanchoe ornamental plant selected from the group consisting of Kalanchoe adelae, Kalanchoe arborescens, Kalanchoe beauverdii, Kalanchoe beharensis, Kalanchoe bentii, Kalanchoe blossfeldiana, Kalanchoe bouvetii, Kalanchoe bracteata, Kalanchoe campanulata, Kalanchoe crenata, Kalanchoe crundallii, Kalanchoe daigremontiana, Kalanchoe delagoensis, Kalanchoe dinklagei, Kalanchoe eriophylla, Kalanchoe farinacea, Kalanchoe fedtschenkoi, Kalanchoe figuereidoi, Kalanchoe flammea, Kalanchoe gastonis, Kalanchoe glaucescens, Kalanchoe gracilipes, Kalanchoe grandidieri, Kalanchoe grandiflora, Kalanchoe hildebrantii, Kalanchoe jongmansii, Kalanchoe kewensis, Kalanchoe laciniata, Kalanchoe laetivirens, Kalanchoe lateritia, Kalanchoe laxiflora, Kalanchoe linearifolia, Kalanchoe longiflora, Kalanchoe luciae, Kalanchoe macrochlamys, Kalanchoe manginii, Kalanchoe marnieriana, Kalanchoe marmorata, Kalanchoe millottii, Kalanchoe miniata, Kalanchoe nyikae, Kalanchoe obtusa, Kalanchoe orgyalis, Kalanchoe peltata, Kalanchoe petitiana, Kalanchoe pinnata, Kalanchoe porphyrocalyx, Kalanchoe prolifera, Kalanchoe pubescens, Kalanchoe pumila, Kalanchoe quartiniana, Kalanchoe rhombopilosa, Kalanchoe robusta, Kalanchoe rolandi, Kalanchoe rosei, Kalanchoe rotundifolia, Kalanchoe schizophylla, Kalanchoe serrata, Kalanchoe sexangularis, Kalanchoe streptantha, Kalanchoe suarezensis, Kalanchoe synsepala, Kalanchoe synsepala f dissecta, Kalanchoe thyrsiflora, Kalanchoe tomentosa, Kalanchoe tubiflora, Kalanchoe uniflora, Kalanchoe velutina, Kalanchoe viguieri, and hybrids thereof.

3. The ornamental plant according to claim 1, wherein said first ornamental plant is obtained by at least 3 generations of inbred breeding.

4. The ornamental plant according to claim 1, wherein said compacted plant growth is compactness expressed as one or more of plant height, inflorescence length, and plant width.

5. The ornamental plant according to claim 4, wherein said plant height, inflorescence length, and plant width is reduced in each inbred generation (Fₓ) as compared to the previous inbred generation (Fₓ₋₁), wherein x is at least 2.

6. The ornamental plant according to claim 5, wherein the ratio between Fₓ and Fₓ₋₁(Fₓ/Fₓ₋₁) of plant height, inflorescence length, and/or plant width is <1.

7. A method for providing an ornamental plant displaying compacted plant growth without use of plant growth regulators during cultivation comprising:
a) vegetatively propagating a first ornamental plant displaying compacted plant growth without use of plant growth regulators during cultivation, said first ornamental plant obtained by inbred breeding of at least 2 generations of a second ornamental plant not displaying compacted plant growth without use of plant growth regulators during cultivation.

8. The method according to claim 7, wherein inbred breeding comprises breeding through selfing.

9. The method according to claim 7 comprising at least 3 generations of inbred breeding.

10. (canceled)

11. A Kalanchoe plant displaying compacted plant growth without use of plant growth regulators during cultivation obtained by inbred breeding of at least 2 generations of a Kalanchoe plant not displaying compacted plant growth without use of plant growth regulators during cultivation; said Kalanchoe plant has a heterozygosity percentage being decreased by at least 20% as compared to the heterozygosity percentage of said second Kalanchoe plant not displaying compacted plant growth without use of plant growth regulators during cultivation.

12. The Kalanchoe plant according to claim 11, wherein said Kalanchoe plant is selected from the group consisting of Kalanchoe adelae, Kalanchoe arborescens, Kalanchoe beauverdii, Kalanchoe beharensis, Kalanchoe bentii, Kalanchoe blossfeldiana, Kalanchoe bouvetii, Kalanchoe bracteata, Kalanchoe campanulata, Kalanchoe crenata, Kalanchoe crundallii, Kalanchoe daigremontiana, Kalanchoe delagoensis, Kalanchoe dinklagei, Kalanchoe eriophylla, Kalanchoe farinacea, Kalanchoe fedtschenkoi, Kalanchoe figuereidoi, Kalanchoe flammea, Kalanchoe gastonis, Kalanchoe glaucescens, Kalanchoe gracilipes, Kalanchoe grandidieri, Kalanchoe grandiflora, Kalanchoe hildebrantii, Kalanchoe jongmansii, Kalanchoe kewensis, Kalanchoe laciniata, Kalanchoe laetivirens, Kalanchoe lateritia, Kalanchoe laxiflora, Kalanchoe linearifolia, Kalanchoe longiflora, Kalanchoe luciae, Kalanchoe macrochlamys, Kalanchoe manginii, Kalanchoe marnieriana, Kalanchoe marmorata, Kalanchoe millottii, Kalanchoe miniata, Kalanchoe nyikae, Kalanchoe obtusa, Kalanchoe orgyalis, Kalanchoe peltata, Kalanchoe petitiana, Kalanchoe pinnata, Kalanchoe porphyrocalyx, Kalanchoe prolifera, Kalanchoe pubescens, Kalanchoe pumila, Kalanchoe quartiniana, Kalanchoe rhombopilosa, Kalanchoe robusta, Kalanchoe rolandi, Kalanchoe rosei, Kalanchoe rotundifolia, Kalanchoe schizophylla, Kalanchoe serrata, Kalanchoe sexangularis, Kalanchoe streptantha, Kalanchoe suarezensis, Kalanchoe synsepala, Kalanchoe synsepala f dissecta, Kalanchoe thyrsiflora, Kalanchoe tomentosa, Kalanchoe tubiflora, Kalanchoe uniflora, Kalanchoe velutina, Kalanchoe viguieri, and hybrids thereof.

13. The Kalanchoe plant according to claim 11, wherein said Kalanchoe plant is obtained by at least 3 generations of inbred breeding.

14. (canceled)

15. The ornamental plant according to claim 1, wherein said first ornamental plant is obtained by at least 7 generations of inbred breeding.

16. The method according to claim 7, comprising at least 7 generations of inbred inbreeding.

17. The Kalanchoe plant according to claim 11, wherein said Kalanchoe plant is obtained by at least 7 generations of inbred breeding.
